(19) United States
(12) Patent Application Publication
(10) Pub. No.: US 2022/0323004 A1
(43) Pub. Date: Oct. 13, 2022

(54) METHODS OF ENHANCING CAR T CELL THERAPY

(71) Applicant: H. LEE MOFFITT CANCER CENTER AND RESEARCH INSTITUTE, INC., Tampa, FL (US)

(72) Inventors: Yoganand BALAGURUNATHAN, Tampa, FL (US); Frederick Lundry LOCKE, Lutz, FL (US); Erin DEAN, Tampa, FL (US)

(21) Appl. No.: 17/638,363

(22) PCT Filed: Aug. 27, 2020
(86) PCT No.: PCT/US20/48120

§ 371 Date: Feb. 25, 2022

Related U.S. Application Data
(60) Provisional application No. 62/892,292, filed on Aug. 27, 2019.

(51) Int. Cl. A61B 5/00; A61B 6/03; A61K 35/17

(52) U.S. Cl. CPC A61B 5/4848; A61B 6/037; A61K 35/17

(57) ABSTRACT

An example method for quantitatively predicting a cancer patient's response to immune-based or targeted therapy and methods of treatment are described herein. In one aspect, disclosed herein are methods for assaying a cancer patient's response to immune-based or targeted therapy, comprising: measuring tumor burden, such as, measuring total metabolic tumor volume either manually or automatically, prior to administration of the immune-based or targeted therapy to create a baseline tumor burden; wherein a high baseline tumor burden indicates that the patient will have a decreased, less efficacious, and/or less durable response, response to immune-based or targeted therapy.

This application claims the benefit of U.S. Provisional Application No. 62/892,292, filed on Aug. 27, 2019, which is incorporated herein by reference in its entirety.

This invention was made with Government support under Grant No. K23-CA201594 awarded by the National Institutes of Health/National Cancer Institute. The Government has certain rights in the invention.

### I. BACKGROUND

Positron emission tomography (PET) has emerged as an important molecular imaging technique to evaluate and manage advance cancer progression, like large B-cell lymphomas. The sensitivity and specificity of 18 fluorodeoxyglucose (FDG) PET is acceptable in the community and has been used for disease staging, monitoring treatment benefits and following recurrence. It has been shown that standardized uptake value (SUV) of some primary tumors computed on FDG PET has shown to have prognostic value. Higher contrast in FDG PET images enabled us the measurement of the metabolic tumor volume (MTV) and more recently Total Tumor Metabolic Volume (TMV) has been investigated as both a prognostic and predictive marker in lymphoma. TMV has been investigated in a wide variety of lymphoma subtypes and disease states (early/relapse/refractory). The total metabolic tumor volume has been evaluated as a predictive marker in assessing disease response to treatment and as a prognostic marker for initial disease burden. However, neither the predictive or prognostic capacity of pre-treatment TMV has been validated in a large prospective studies.

Axicabtagene ciloleucel (axi-cel) is an anti-CD19 targeted Chimeric Antigen Receptor (CAR) T-cell therapy for large B-cell lymphoma (LBCL). High tumor burden, by sum of the product of diameters (SPD) in ≥6 reference lesions, was associated with lower durable responses rates in the ZUMA-1 trial. Given limitations of SPD as a measure of tumor burden, what are needed are new methods of assaying the efficacy of CAR T cell therapy.

### II. SUMMARY

Disclosed are methods related to immune-based or targeted therapy.

In one aspect, disclosed herein are methods for assaying a cancer patient's response to immune-based or targeted therapy, comprising: measuring tumor burden (such as, measuring total metabolic tumor volume either manually or automatically) prior to administration of the immune-based or targeted therapy to create a baseline tumor burden; wherein a high baseline tumor burden (for example, >130 mL) indicates that the patient will have a decreased, less efficacious, and/or less durable response. response to immune-based or targeted therapy.

Also disclosed herein are methods of assaying a cancer patient's response to immune-based or targeted therapy of any preceding aspect, wherein tumor burden is measured 1, 2, 3, 4, 5, 6, 7, 8, 9, 10, 11, 12, 13, 14, 15, 16, 17, 19, 20, 21, 22, 23, 24, 25 or more additional times after administration of the immune-based or targeted therapy.

In one aspect, disclosed herein are methods of assaying a cancer patient's response to immune-based or targeted therapy of any preceding aspect, wherein total metabolic tumor volume measures glucose uptake. For instance, the total metabolic tumor volume is measured either manually or by functional imaging that measures glucose uptake (such as, for example by positron emission tomography (PET) including, but not limited to, 18 fluorodeoxyglucose (FDG) PET)). In one aspect the tumor burden is not assayed by sum of the product diameters (SPD).

Also disclosed herein are methods of assaying a cancer patient's response to immune-based or targeted therapy of any preceding aspect, wherein the immune based or targeted therapy comprises an immunodepleting therapy and a CAR T cell infusion (such as, for example, an anti-CD19 CAR T cell infusion).

In one aspect, disclosed herein are methods of treating, inhibiting, reducing, decreasing, ameliorating and/or preventing a cancer and or metastasis (including a cancer recurrence) in a subject comprising measuring tumor burden (such as, for example, total metabolic tumor volume either manually, semi-automatically, or automatically) prior to administration of the immune-based or targeted therapy to create a baseline tumor burden; wherein a high baseline tumor burden (for example, >130 mL) indicates that the patient will have a decreased, less efficacious, and/or less durable response to immune-based or targeted therapy relative to a control; wherein a low baseline tumor burden a patient (≤130 mL) will have an efficacious response to immune-based or targeted therapy relative to a control; and administering to a patient with a low baseline tumor burden an immune-based or targeted therapy or administering to a patient with a high baseline tumor burden a chemotherapeutic agent, a higher dose of immune-based or targeted therapy, or multiple rounds of immune-based or targeted therapy.

Also disclosed herein are methods of methods of treating, inhibiting, reducing, decreasing, ameliorating and/or preventing a cancer and or metastasis (including a cancer recurrence) in a subject with a cancer of any preceding aspect, further comprising measuring tumor burden 1, 2, 3, 4, 5, 6, 7, 8, 9, 10, 11, 12, 13, 14, 15, 16, 17, 19, 20, 21, 22, 23, 24, 25 or more additional times following administration of the immune-based or targeted therapy.

In one aspect, disclosed herein are methods of treating, inhibiting, reducing, decreasing, ameliorating and/or preventing a cancer and or metastasis (including a cancer recurrence) in a subject with a cancer of any preceding aspect, wherein total metabolic tumor volume measures glucose uptake. For instance, the total metabolic tumor volume is measured either manually or by functional imaging that measures glucose uptake (such as, for example by positron emission tomography (PET) including, but not limited to, 18 fluorodeoxyglucose (FDG) PET)). In one aspect the tumor burden is not assayed by sum of the product diameters (SPD).

Also disclosed herein are methods of treating, inhibiting, reducing, decreasing, ameliorating and/or preventing a cancer and or metastasis (including a cancer recurrence) in a subject with a cancer of any preceding aspect, wherein the immune based or targeted therapy comprises an immunodepleting therapy and a CAR T cell infusion (such as, for example, an anti-CD19 CAR T cell infusion).

In one aspect, disclosed herein are methods of treating, inhibiting, reducing, decreasing, ameliorating and/or preventing a cancer and or metastasis (including a cancer recurrence) in a subject of any preceding aspect, further comprising debulking the tumor in the subject to a low baseline tumor volume and administering to the subject an immune-based or targeted therapy after a low baseline tumor burden is reached.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, and 1C show baseline PET images of a 71-year-old woman with refractory double hit diffuse LBCL. Patient has 1 actively metabolic tumor lesion. MTV 33 mL.

FIG. 2 shows PET image of a 60-year-old man with multiple tumors, a fraction of which are displayed below as automatically generated MIM lesions. MTV 411 mL.

FIG. 3 shows Kaplan-Meier survival curves by MTV.

FIG. 4 shows the response rates to axi-cel by MTV.

FIG. 5 shows a comparison of MTV (n=48) and MTV_AUTO (n=48) to SPD (n=35).

FIG. 6 shows an example of PET-CT and standardized uptake value (SUVmax).

FIG. 7 shows an example of semantic feature contour.

FIG. 8 shows an example of semantic features fusion, diaphragm, and symmetry.

FIG. 9 shows an example of semantic features fusion, diaphragm, and symmetry.

FIG. 10 shows an example of CT semantic features border, infiltration, and adiposity.

FIG. 11 shows an example of CT semantic features border, infiltration, and adiposity.

FIGS. 12A and 12B show an example of a case of MTV calculations in a patient via the two investigated MTV estimation methods. PET images in the (12A) MTV-Semi-Automated method and (12B) MTV-Manual method with tumor outlined in blue.

FIGS. 13A, 13B, 13C, and 13D show Kaplan-Meier survival curves and log-rank p-values by low versus high MTV-Manual (cut-off 147.5 mL). (13A) OS (13B) PFS for patients in cohort 1 (n=48). (13C) OS (13D) PFS for patients in cohort 2 (n=48).

FIGS. 14A and 14B show Kaplan-Meier survival curves by low versus high MTV-Manual (cut-off 147.5 mL). (14A) OS (14B) PFS for patients in cohort 1 and 2 combined (n=96).

FIGS. 15A and 15B show Kaplan-Meier survival curves by low versus high MTV-Manual (cut-off 147.5 mL) quartiles. (15A) OS (15B) PFS for patients in cohort 1 and 2 combined (n=96).

FIGS. 16A and 16B show response rates to axi-cel by low versus high MTV-Manual (cut-off 147.5 mL). (16A) ORR (16B) CR for both cohorts combined (n=96).

FIGS. 17A, 17B, 17C, and 17D show toxicities to axi-cel by low versus high MTV-Manual (cut-off 147.5 mL). (17A) Any NT (17B) G3-4 NT (17C) Any CRS (17D) G3-4 CRS for both cohorts combined (n=96).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity.

Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method of Treating Cancer and Assessing Efficacy of Immunotherapy

There is currently no standard for calculating tumor burden in lymphoma. For ZUMA-1, tumor burden was evaluated by the SPD of bi-directional measurements in up to 6 reference lesions on computed tomography. Alternative methods include calculating maximum standardized uptake value (SUVmax) or baseline metabolic tumor volume (MTV) on fluorine-18 fluorodeoxyglucose positron emission tomography/computed tomography (¹⁸F-FDG PET/CT). SUVmax is a semiquantitative measurement of glucose metabolism that has been associated with clinical outcomes in malignant lymphomas. It is based on a single-pixel on the scan, which represents the maximum intensity of ¹⁸F-FDG activity in the tumor. Similar to SPD, SUVmax is not a direct three-dimensional measure of tumor and only serves as an approximation of tumor burden.

MTV is of special interest since it has the potential to capture all metabolically active, and therefore presumably malignant, areas within a tumor mass and across the body for an accurate tumor burden determination. It has been shown to be prognostic in non-Hodgkin's lymphoma, with high MTV associated with worse outcomes following immunotherapy, chemotherapy, or radiation therapy. The clinical significance of baseline tumor burden as determined by MTV in CAR T-cell treated lymphoma patients, however, remains unclear. Therefore, this study aimed at assessing the relationship between baseline MTV and survival, including overall survival (OS) and progression free survival (PFS), among axi-cel treated patients. Evaluating the relationship between baseline MTV and response rates and toxicity were secondary objectives. Optimizing MTV calculation and comparing different tumor burden estimates were additionally investigated.

As noted herein, high metabolic tumor burdens were associated with poor patient outcomes and low metabolic tumor volumes were associated with successful outcomes. In one aspect, disclosed herein are methods for assaying a cancer patient's response to immune-based or targeted therapy (such as, for example a chimeric antigen receptor (CAR) T cell therapy, including but not limited to anti-CD19 CAR T cells such as, for example, axi-cel), comprising: measuring tumor burden (such as, measuring total metabolic tumor volume either manually or automatically) prior to administration of the immune-based or targeted therapy to create a baseline tumor burden; wherein a high baseline tumor burden (for example, >130 mL) indicates that the patient will have a decreased, less efficacious, and/or less durable response. response to immune-based or targeted therapy.

It is understood and herein contemplated that the disclosed methods of assaying a cancer patient's response to immune-based or targeted therapy comprise obtaining primary patient data (such as tumor burden as determined by metabolic tumor volume) at multiple timepoints prior to administration of an anti-cancer therapy (such as, for example, immune-based or targeted therapy). In one aspect the primary patient data (such as, for example, tumor burden) is measured at one or more timepoints such as, for example, at 1, 2, 3, 4, 5, 6, 7, 8, 9, 10, 11, 12, 13, 14, 15, 16, 17, 18, 19, 20, 21, 22, 23, 24, 28, 30, 32, 36, 40, 42, 44, or 48 hours, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 40, 45, 50, 55, 60, 65, 70, 75, 80, 85, or 90 days prior to administration of an anti-cancer therapy (such as, for example an immune-based or targeted therapy including, but not limited to CAR T cell therapy or any other immune-based or targeted therapy disclosed herein).

As noted herein, the disclosed methods of assaying a cancer patient's response to immune-based or targeted therapy comprise measuring total metabolic tumor volume and assessing the efficacy of the therapy based on the total metabolic tumor volume where a high metabolic tumor volume is less efficacious than a low tumor volume. In one aspect, the cutoff of a low or high tumor volume is between 130-170 mL, more preferably between 140-160 mL, most preferably between 145 and 155 mL. For example, the cutoff can be 130, 131, 132, 133, 134, 135, 136, 137, 138, 139, 140, 141, 142, 143, 144, 145, 146, 147, 147.5, 148, 149, 150, 151, 152, 153, 154, 155, 156, 157, 158, 159, 160, 161, 162, 163, 164, 165, 166, 167, 168, 169, or 170 mL. In one aspect a low metabolic tumor volume is ≤130, ≤140, ≤145, ≤147.5, ≤150, ≤155 mL. Stated differently, in one aspect, a high tumor metabolic tumor volume is >130, >140, >145, >147.5, >150, >155 mL.

It is understood and herein contemplated that the disclosed methods of measuring total metabolic tumor volume can be performed manually, semi-automatically, or automatically utilizing any method for that accounts for/measures glucose uptake. Thus, MTV can be measured manually or using functional imaging that measures glucose uptake such as positron emission tomography (PET) including, but not limited to, 18 fluorodeoxyglucose (FDG) PET). It is understood and herein contemplated that measurements such as sum of the product diameters (SPD) do not take into account glucose uptake and thus would not be appropriate for use in the disclosed methods. Accordingly, in one aspect, disclosed herein are methods of assaying a cancer patient's response to immune-based or targeted therapy, wherein total metabolic tumor volume measures glucose uptake. For instance, the total metabolic tumor volume is measured either manually or by functional imaging that measures glucose uptake (such as, for example by positron emission tomography (PET) including, but not limited to, 18 fluorodeoxyglucose (FDG) PET)). In one aspect the tumor burden is not assayed by sum of the product diameters (SPD).

It is understood and herein contemplated that while being able to determine or predetermine if an immune-based therapy or targeted therapy will be successful is very useful, but the ultimate goal is to use this information to provide a treatment regimen that will provide a successful outcome. Thus, in one aspect, disclosed herein are methods of treating, inhibiting, reducing, decreasing, ameliorating and/or preventing a cancer and or metastasis (including a cancer recurrence) in as subject comprising measuring tumor burden (such as, for example, measuring total metabolic tumor volume either manually or automatically) prior to administration of the immune-based or targeted therapy to create a baseline tumor burden; wherein a high baseline tumor burden (for example, >130 mL) indicates that the patient will have a decreased, less efficacious, and/or less durable response to immune-based or targeted therapy relative to a control; wherein a low baseline tumor burden a patient (for example, ≤130 mL) will have an efficacious response to immune-based or targeted therapy relative to a control; and administering to a patient with a low baseline tumor burden an immune-based or targeted therapy or administering to a patient with a high baseline tumor burden a chemotherapeutic agent, a higher dose of immune-based or targeted therapy, or multiple rounds of immune-based or targeted therapy.

In one aspect, the disclosed herein are methods of treating, inhibiting, reducing, decreasing, ameliorating and/or preventing a cancer and or metastasis (including a cancer recurrence) in a subject comprise obtaining primary patient data (such as tumor burden) at multiple timepoints prior to administration of an anti-cancer therapy (such as, for example, immune-based or targeted therapy). In one aspect the primary patient data (such as, for example, tumor burden) is measured at one or more timepoints such as, for example, at 1, 2, 3, 4, 5, 6, 7, 8, 9, 10, 11, 12, 13, 14, 15, 16, 17, 18, 19, 20, 21, 22, 23, 24, 28, 30, 32, 36, 40, 42, 44, or 48 hours, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 40, 45, 50, 55, 60, 65, 70, 75, 80, 85, or 90 days prior to administration of an anti-cancer therapy (such as, for example an immune-based or targeted therapy including, but not limited to CAR T cell therapy).

As noted herein, the disclosed methods of treating, inhibiting, reducing, decreasing, ameliorating and/or preventing a cancer and or metastasis (including a cancer recurrence) comprise measuring total metabolic tumor volume and assessing the efficacy of the therapy based on the total metabolic tumor volume where a high metabolic tumor volume is less efficacious than a low tumor volume. In one aspect, the cutoff of a low or high tumor volume is between 130-170 mL, more preferably between 140-160 mL, most preferably between 145 and 155 mL. For example, the cutoff can be 130, 131, 132, 133, 134, 135, 136, 137, 138, 139, 140, 141, 142, 143, 144, 145, 146, 147, 147.5, 148, 149, 150, 151, 152, 153, 154, 155, 156, 157, 158, 159, 160, 161, 162, 163, 164, 165, 166, 167, 168, 169, or 170 mL. In one aspect a low metabolic tumor volume is ≤130, ≤140, ≤145, ≤147.5, ≤150, ≤155 mL. Stated differently, in one aspect, a high tumor metabolic tumor volume is >130, >140, >145, >147.5, >150, >155 mL.

As noted above, it is understood and herein contemplated that the disclosed methods of measuring total metabolic tumor volume can be performed manually, semi-automatically, or automatically utilizing any method for that accounts for/measures glucose uptake. Thus, MTV can be measured manually or using functional imaging that measures glucose uptake such as positron emission tomography (PET) including, but not limited to, 18 fluorodeoxyglucose (FDG) PET). It is understood and herein contemplated that measurements such as sum of the product diameters (SPD) do not take into account glucose uptake and thus would not be appropriate for use in the disclosed methods. Accordingly, in one aspect, disclosed herein are methods of treating, inhibiting, reducing, decreasing, ameliorating and/or preventing a cancer and or metastasis (including a cancer recurrence) in a subject, wherein total metabolic tumor volume measures glucose uptake. For instance, the total metabolic tumor volume is measured either manually or by functional imaging that measures glucose uptake (such as, for example by positron emission tomography (PET) including, but not limited to, 18 fluorodeoxyglucose (FDG) PET)). In one aspect the tumor burden is not assayed by sum of the product diameters (SPD).

In one aspect, it is understood that a subject can need ongoing therapy or subsequent administrations of an anti-cancer therapy. Thus, in one aspect, disclosed herein are methods of treating, inhibiting, reducing, decreasing, ameliorating and/or preventing a cancer and or metastasis (including a cancer recurrence), wherein the method further comprises obtaining primary patient data (such as assaying tumor burden) at one or more time points following administration of an anti-cancer therapy (such as, for example an immune-based or targeted therapy including, but not limited to CAR T cell therapy) at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 36, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, or 365 days; 1, 2, 3, 4, 5, 6, 7, 8, 9, 10, 11, 12, 13, 14, 15, 16, 17, 18, 19, 20, 21, 22, 23, 24, 25, 26, 28, 32, 36, 40, 44, 48, or 52 weeks; or 1, 2, 3, 4, 5, 6, 7, 8, 9, 10, 11, 12, 13, 14, 15, 16, 17, 18, 19, 20, 21, 22, 23, 24 months post administration of the immune-based or targeted therapy.

In one aspect, the immune based or targeted therapy comprises immunodepletion followed by administration of a CAR T cells (i.e., CAR T cell infusion). The CAR T cells can come from any immunocomatible source including, but not limited to autologous CAR T cells. The CAR T cells can be designed to target any tumor antigen known in the art including, but not limited to a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-ab1, HER2, β-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TES1, PAX5, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-1a, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRCSD, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin.

The disclosed compositions can be used to treat any disease where uncontrolled cellular proliferation occurs such as cancers. A non-limiting list of different types of cancers that the disclosed methods can be used to treat is the following: lymphoma, B cell lymphoma (including large B cell lymphomas such as, for example, diffuse large B cell lymphomas), T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

It is understood and herein contemplated that the immune-based or targeted therapy can include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate) as well as not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Given the difference in response that metabolic tumor volume has on the outcome or immune based or targeted immunotherapies, when a high metabolic tumor volume is measured, one alternative treatment step can comprise debulking the tumor (for example, by lowering the metabolic tumor volume by physical, radiological, or chemical means) in the subject prior to the onset of any treatment and then, after a low metabolic tumor volume is attained, administering to the subject n immune-based or targeted therapy.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).
a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.
b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone can range from about 1 μg/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Association of High Baseline Metabolic Tumor Volume with Response Following Axicabtagene Ciloleucel in Refractory Large B-Cell Lymphoma

### a) Methods

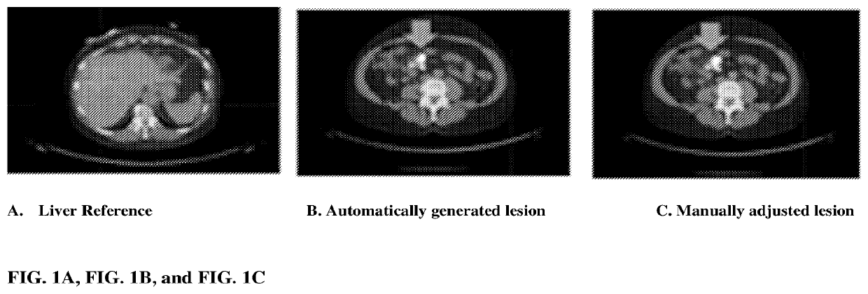

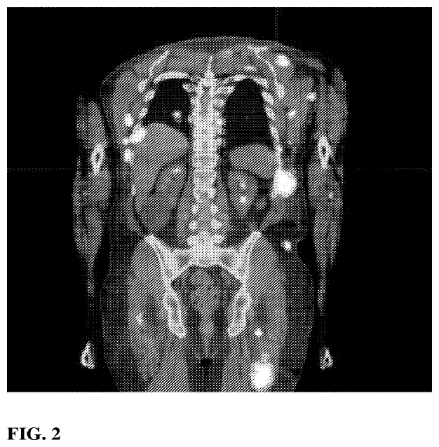

Non-tumor lesions were manually removed and missed tumor lesions manually added. MTV was measured by MIM Software using a 41% SUVmax threshold with manual lesion contour adjustment and radiologist review (FIGS. 1 and 2). Low and high MTV groups were defined based on median cutoff value. SPD values were recorded upon chart review from the clinical radiologist's report. Cytokine release syndrome (CRS) was graded. Neurotoxicity (NT) was graded by CTCAEv4. Toxicities, overall response rate (ORR), and complete response rate (CR) were evaluated via Fisher's test; PFS and OS via Kaplan-Meier and log-rank test. MTV on baseline 18F-FDG PET/CT scans was measured by MIM Software (MIM Software Inc, Cleveland, Ohio) using a 41% SUVₘₐₓ threshold workflow function. MTV and MTV_AUTO were compared to SPD via linear regression and correlation.

### b) Results

48 patients with LBCL, or its variants, that received axi-cel at Moffitt from June 2015 to October 2018 were included. 31 were male, and median age was 63 years (range, 28-76)(Table 1, cohort 1). CRS occurred in 45/48 (94%) and NT in 32/48 (67%) patients. Grade 3-4 CRS in 2/48 (4%) and NT in 12/48 (25%).

TABLE 1
Baseline patient characteristics at the
time of axi-cel infusion and clinical outcomes with axi-cel.

| | Cohort 1, n = 48 | Cohort 2, n = 48 | |
|---|---|---|---|
| | (%) | (%) | P value |
| Characteristic | | | |
| Age (years) | | | |
| Median, range | 63, 28-76 | 64, 19-79 | 0.83 |
| Gender | | | |
| Male | 31 (65) | 30 (62.5) | 0.83 |
| ECOG (0-5) | | | |
| 0-1 | 41 (86) | 37 (77) | 0.29 |
| 2-3 | 7 (14) | 11 (23) | |
| Histology | | | |
| Diffuse large B-cell lymphoma (DLBCL), not | 26 (54) | 21 (44) | 0.25 |
| otherwise specified (NOS) | | | |
| Germinal center B-cell like (GCB) | 10 (21) | 10 (21) | 1.00 |
| [cases with unknown Double hit/Triple hit status] | [1] | [4] | |
| Activated B-cell like (ABC) | 13 (27) | 8 (17) | 0.23 |
| [cases with unknown Double hit/Triple hit status] | [2] | [2] | |
| Unknown if GCB or ABC | 3 (6) | 3 (6) | 1.00 |
| [cases with unknown Double hit/Triple hit status] | [0] | [1] | |
| High grade B-cell lymphoma, with MYC and | 7 (15) | 8 (17) | 0.79 |
| BCL2 and/or BCL6 rearrangements | | | |
| B-cell lymphoma, unclassifiable, with features | 12 (25) | 17 (35) | 0.28 |
| intermediate between DLBCL and classical | | | |
| Hodgkin lymphoma | | | |
| Primary mediastinal B-cell lymphoma | 3 (6) | 2 (4) | 0.65 |
| Stage (I-IV) | | | |
| I/II | 10 (21) | 12 (25) | 0.62 |
| III/IV | 38 (79) | 36 (75) | |
| LDH Level before conditioning > 2xULN | 11 (23) | 9 (19) | 0.61 |
| Yes | | | |
| Extranodal sites > 1 | | | |
| Yes | 24 (50) | 33 (69) | 0.06 |
| Secondary R-IPI score (1-5) | | | |
| 0 | 1 (2) | 2 (4) | 0.50 |
| 1-2 | 18 (38) | 12 (25) | |
| 3-5 | 26 (54) | 32 (67) | |
| N/A (Primary mediastinal B-cell | 3 (6) | 2 (4) | |
| lymphoma) | | | |
| Prior lines of therapy | | | |
| Median, range | 3, 2-7 | 3, 2-8 | 0.83 |
| Bridging Therapy | | | |
| Yes | 15 (31) | 31 (65) | 0.01 |
| Chemotherapy/targeted therapy | 2 (4) | 10 (21) | |
| Steroids | 5 (10) | 4 (8) | |
| Radiation therapy | 1 (2) | 4 (8) | |
| Combination chemotherapy/targeted | 7 (15) | 13 (27) | |
| therapy +/− steroids +/− radiation | | | |
| therapy | | | |
| Received prior to baseline PET/CT | 7 (15) | 17 (35) | 0.02 |
| Axi-cel Administration | | | |
| Trial (Zuma-1) | 22 (46) | 0 (0) | <0.0001 |
| Commercial (Yescarta) | 26 (54) | 48 (100) | |
| Outcome | | | |
| Clinical Response to axi-cel | | | |
| CR by last follow up | 32 (67) | 31 (65) | 0.83 |
| ORR by last follow up | 39 (81) | 35 (73) | 0.33 |
| ORR at 3 months | 26 (54) | 25/47 (53) | |
| ORR at 6 months | 23 (48) | 20/47 (43) | |
| ORR at 12 months | 21 (44) | | |
| Follow up and Survival | | | |
| Median follow-Up for survivors in | 24.98 (10.59- | 12.03(0.89-25.74) | 0.00 |
| months (range) | 51.02) | | |
| Median OS in months (95% CIs) | 34.98 (14.33- | Not reached | |
| | 34.98) | | |
| Median PFS in months (95% CIs) | 5.85 (2.99-11.54) | 13.84 (3.95- | 0.54 (Log rank) |
| | | 13.84) | |
| Toxicity, Grade (G) 0-5 | | | |
| CRS | 45 (94) | 45 (94) | 1.00 |
| G3-4 CRS | 2 (4) | 7 (15) | 0.08 |
| NT | 32 (67) | 35 (73) | 0.50 |
| G3-4 NT | 12 (25) | 16 (33) | 0.37 |

As shown in Table 2, median follow up for survivors was 8.9 months (range, 1.4-36.8 months). CR was achieved in 31/48 (64.6%) and ORR in 39/48 (81.3%). Median for the low MTV group was 35.1 mL (range, 4.24-132.8 mL), and for the high MTV group 455.5 mL (range, 162.2-1221.4 mL).

TABLE 2
Follow up, cutoff MTV, response rates and toxicities

| Median follow up for survivors | 8.9 months (range, 1.4-36.8 months) |
|---|---|
| Median cutoff MTV | 147.5 mL |
| ORR | 39/48 (81.3%) |
| CR | 31/48 (64.6%) |
| CRS | 45/48 (93.8%) |
| Grade 3-4 CRS | 2/48 (4.2%) |
| NT | 32/48 (66.7%) |
| Grade 3-4 NT | 12/48 (25%) |

High MTV was not predictive of G1-4 NT or G3-4 NT (OR = 1.00, P = 0.99; OR = 1.56, P = 0.74) Similarly, high MTV was not predictive of G1-4 CRS or G3-4 CRS (OR = 0.47, P = 0.99; OR = 1.00, P = 0.99)

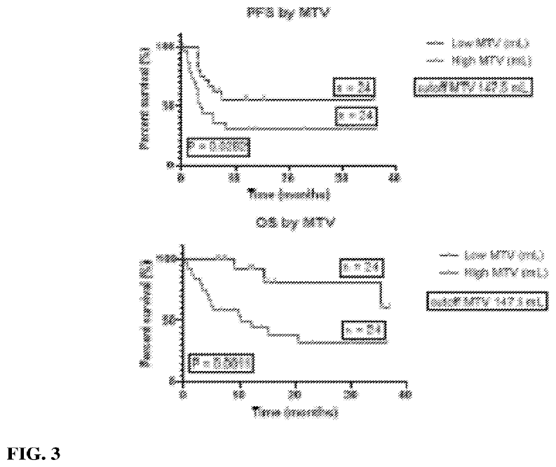

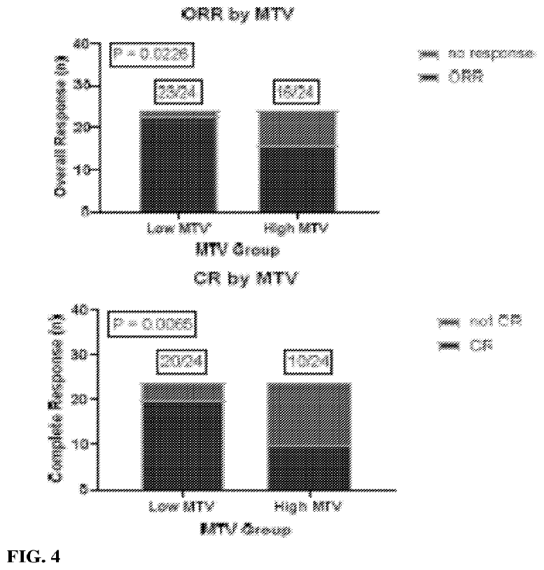

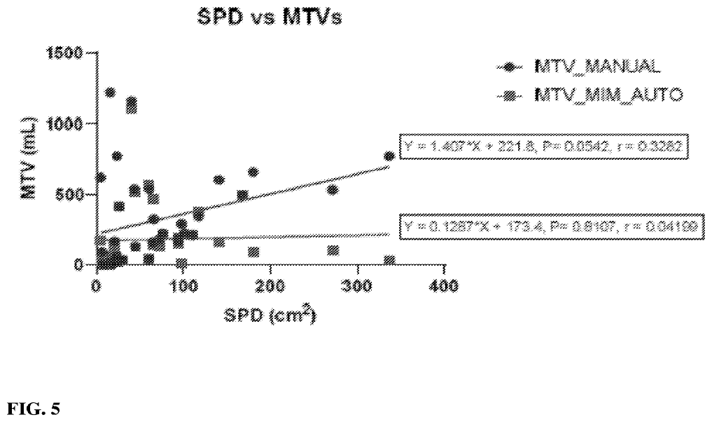

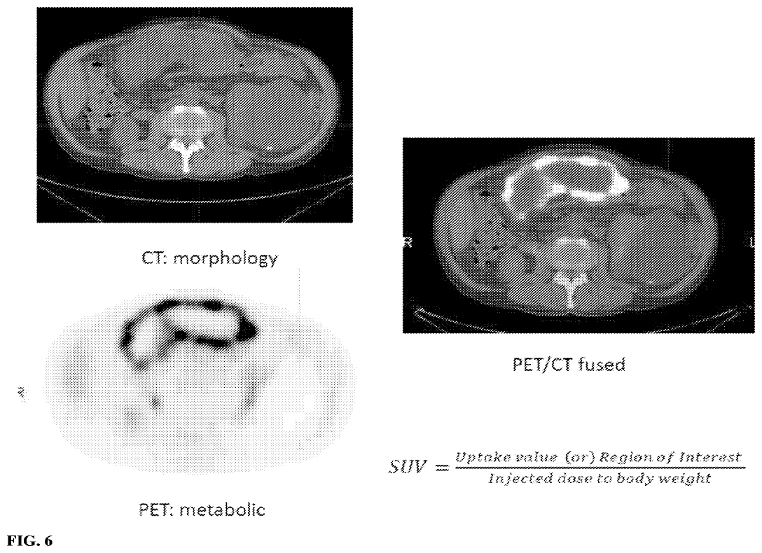

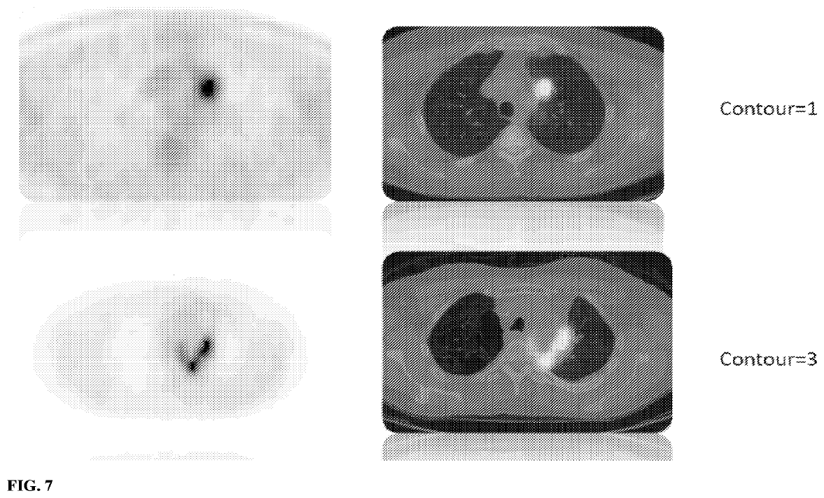

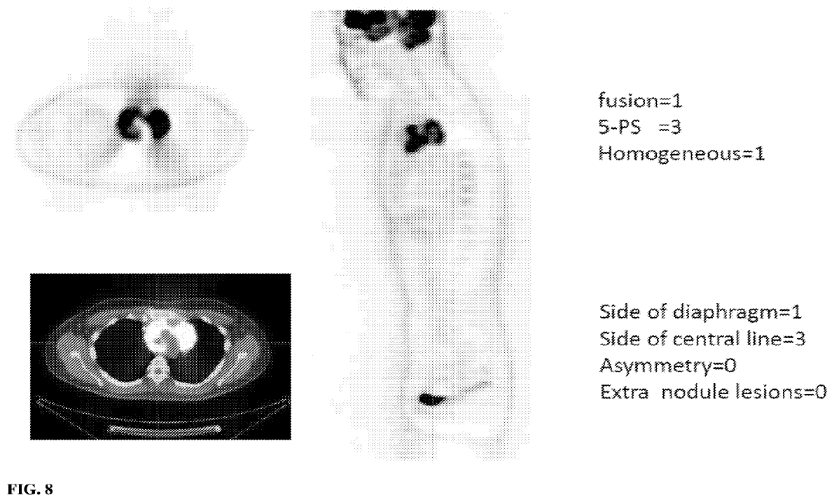

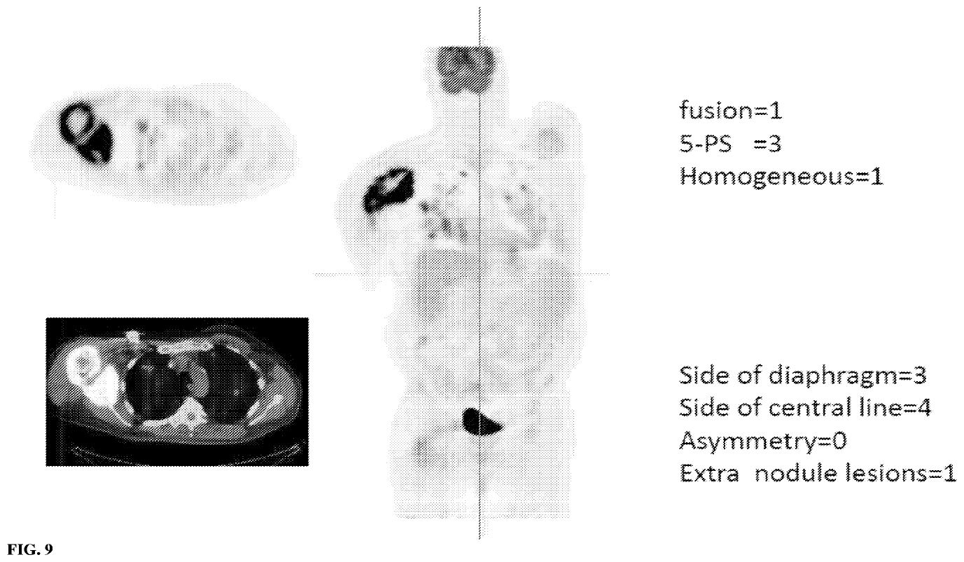

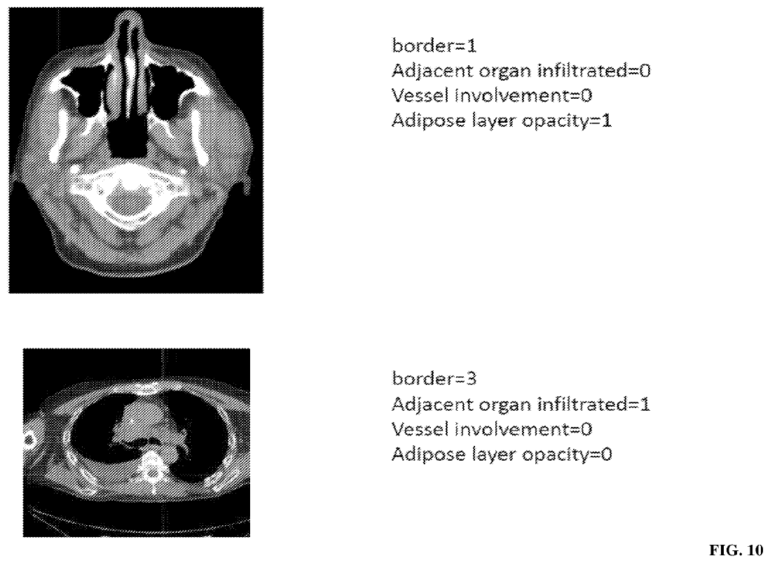

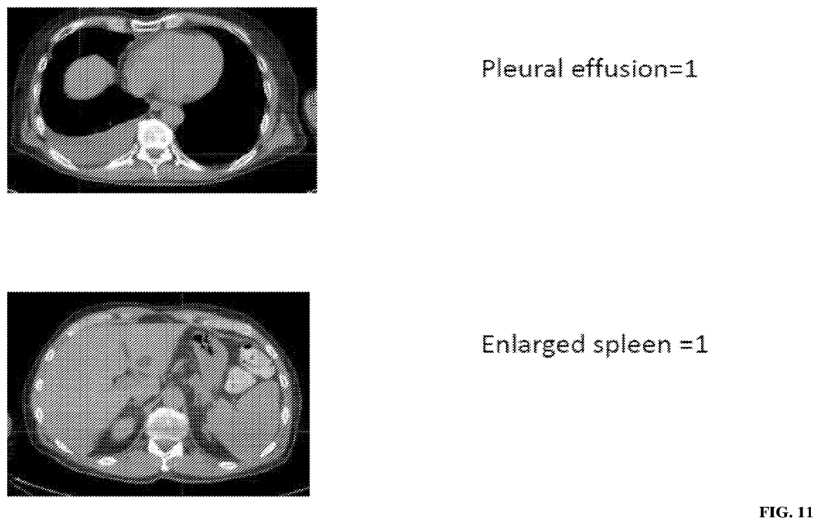

High MTV was not predictive of G1-4 NT or G3-4 NT (OR=1.14, P=0.99; OR=1.66, P=0.52). Similarly, high MTV was not predictive of G1-4 CRS or G3-4 CRS (OR=0.29, P=0.348; OR=1.05, P=0.99). Low MTV was predictive of ORR (OR=11.50, P=0.026) and CR (OR=9.8, P=0.002). Patients with high MTV had inferior PFS (HR=3.296, 95% CI 1.42-7.64, P=0.008) and OS (HR=6.68, 95% CI 2.56-17.32, P=0.003). FIG. 3 shows Kaplan-Meier survival curves by MTV. FIG. 4 shows the response rates to axi-cel by MTV. FIG. 5 shows a comparison of MTV (n=48) and MTV_AUTO (n=48) to SPD (n=35).

### c) Conclusions

High baseline MTV is associated with decreased and less durable response following axi-cel. While more labor intensive and time consuming, manual baseline MTVs have a better association with SPDs compared to automatically generated MTVs.

### Example 2: Tumor Burden and Radiomics as Surrogate Markers in B-Cell Lymphoma

Positron emission tomography (PET) has emerged as an important molecular imaging technique to evaluate and manage advance cancer progression, like large B-cell lymphomas. The sensitivity and specificity of 18 fluorodeoxyglucose (FDG) PET is acceptable in the community and has been used for disease staging, monitoring treatment benefits and following recurrence. It has been shown that standardized uptake value (SUV) of some primary tumors computed on FDG PET has shown to have prognostic value. Higher contrast in FDG PET images allowed for measurement of the metabolic tumor volume (MTV) and more recently Total Tumor Metabolic Volume (TMV) has been investigated as both a prognostic and predictive marker in lymphoma. TMV has been investigated in a wide variety of lymphoma subtypes and disease states (early/relapse/refractory). The total metabolic tumor volume has been evaluated as a predictive marker in assessing disease response to treatment and as a prognostic marker for initial disease burden. However, neither the predictive or prognostic capacity of pre-treatment TMV has been validated in a large prospective studies.

In the last few years, quantitative imaging has developed expertise in development of (imaging and quantitative) metrics that are used to identify, extraction information on lesions of interest. As shown herein these markers provide additive predictive and prognostic value that enable its use for patient care. Herein is evaluated whether pre-treatment tumor burden, as measured by TMV, predicts for response (PD/SD vs PR/CR), durable response (PR/CR at 1 year), OS and PFS (stratified into high TMV and Low TMV with comparison of KM curves) in patients treated on the ZUMA-1 trial. TMV from the most recent PET done prior to KTE-C19 infusion in all 108 patients that were treated on the phase 1 and phase 2 Zuma-1 study can be calculated. Also shown herein is an assessment of the relationship between initial TMV and G3-5 Neurologic Toxicity or G3-5 CRS.

As a consequence of evaluating tumor burden, as measured by TMV, as a biomarker for response, the changes in tumor burden can also be evaluated over time to inform a mathematical model of CD19 CAR T therapy. Two subsequent PET scans (day +30 and day +90) post KTE-C19 can be evaluated for TMV to understand the dynamics of tumor response over time in all 108 patients. Additionally, the variability of TMV computation to individual versus cluster of tumors in these advanced stage disease can be studied. Additionally, the relationship between MTV (derived on PET) to tumor volume/size and quantitative imaging metrics (Radiomics) derived on CT images can be assessed, as well, as relationships of Tumor size/volume, quantitative imaging metrics (Radiomics) derived on CT images to clinical outcome. Tumoral heterogeneity in the regions observed on PET can be assessed using quantitative imaging metrics (Radiomics). This work leads to the development and ability to relate observational semantic traits (radiologist observed) to disease progression (see Table 3 for PET semantic features and Table 4 for CT semantic features). Lastly, the work herein allows for the development of imaging tools (integrated with commercial PACS or stand-alone) to compute TMV and texture metrics with certain level of radiologist inputs.

TABLE 3
PET semantic Features

| | Score |
|---|---|
| The largest dominant tumor | |
| Contour | 1 = round/oval; 2 = somewhat |
| | irregular; 3 = irregular |
| Fusion | 0 = absence; 1 = presence |
| 5-PS | 1 = PS1; 2 = PS2,3; 3 = PS4,5 |
| Homogenous | 0 = absence; 1 = presence |
| The longest transverse | |
| diameter (LDi) | |
| The shortest axis | |
| perpendicular LDi | |
| SUVmax | |
| All Lesions | |
| Side of diaphragm | 1 = above; 2 = below; 3 = both |
| Sine of Central Line | 1 = left; 2 = right; 3 = central; 4 = both or three |
| Asymmetry | 0 = absence; 1 = presence |
| Extra Nodal Lesions | 0 = absence; 1 = presence |
| The number of involved organs | 1 = 0; 1 = 1-3; 3 = 4 and more |
| The number of lesions | 1 = 1-5; 2 = 6-10; 3 = more than 10 |
| TMV | |

TABLE 4
CT semantic features

| | Score |
|---|---|
| The largest dominant tumor | |
| Border | 1 = well defined; 2 = everything between |
| | 1 and 3; 3 = poorly defined |
| Adjacent organs infiltrated | 0 = absence; 1 = presence |
| Vascular involvement | 0 = absence; 1 = presence |
| Calcification | 0 = absence; 1 = presence |
| Adipose layer opacity | 0 = absence; 1 = presence |
| Associate findings | |
| Enlargement of Spleen | 0 = absence; 1 = presence |
| Pleural effusion | 0 = absence; 1 = presence |

a) Inclusion Criteria:

All 108 ZUMA-1 P1/P2 patients.
b) Exclusion Criteria:

Any patient that the images are not available for TMV assessment.
c) Primary Endpoints:

TMV-Predictor of Outcome in patients treated with CAR-T with respect to PFS/OS.

Find surrogate imaging texture metrics (Radiomics) for TMV and disease progression.
d) Secondary Endpoints:

Assess radiological sematic traits for these patients to progression (PFS/OS).
e) Sample Size Justification/Statistical Analysis:

The TMV of the tumors can be calculated for in a set of 30 patients randomly selected out of the 108 patients, with equal number from three groups: Complete response (CR), Partial response (PR), Non-responder (NR) on ZUMA-1. TMV for these cases can be related to the treatment response of the drug, measured in months as Progression free survival (PFS) sing Kaplan meier plots and significance tested using log-rank test. The odds ratio for pairs of these groups can be computed.

### Example 3

### a) Patients and Methods

### (1) Patient Population

Approval for retrospective review of patient records was obtained from the Institutional Review Board. Ninety-six patients with relapsed or refractory LBCL who received first axi-cel treatment from May 2015 to June 2019 were included. All patients had ¹⁸F-FDG PET/CT scans and clinical data. An initial cohort (cohort 1) of 48 patients was used to create the index association model constructed in 2018. A second test cohort (cohort 2) was created in 2019. Patients previously treated with CAR T-cell therapy, without measurable lesions on imaging, or without baseline PET were excluded. Elevated LDH before lymphodepleting chemotherapy was defined as LDH>2×Upper Limit of Normal (ULN). Bridging therapy was defined as any lymphoma specific therapy given after apheresis, but prior to the start of fludarabine cyclophosphamide chemotherapy for lymphodepletion before CAR T-cell infusion. Patient characteristics data was compiled on 4/19/2020.

### (2) Methodology and Workflow

Baseline MTV was calculated via custom tool implemented on MIM PACS version 6.8.4 (MIM Software Inc, Cleveland, Ohio). Using this workflow function, the right lobe of the liver was first manually selected to create a ≤3 cm spherical volume of interest (VOI) that served as a reference. Using the liver reference, metabolically active regions were identified and displayed on the scan similar to the PET Response Criteria in Solid Tumors (PERCIST criteria). Each lesion had a peak standardized uptake value corrected for lean body mass (SULₚₑₐₖ) equal or greater than the mean SUL in the liver VOI plus two times its standard deviation. Corresponding PET regions were subsequently computed. For each PET region, voxels equal or greater than 41% of SUVmax were identified and a boundary was set to create a metabolically active region. The workflow included voxels over 41% SUVmax to be considered for MTV calculation of the lesion (mL). For imaging studies without abdominal region, an alternative workflow was used to calculate the MTV, which used a previously calculated liver reference for the same patient scan. For ¹⁸F-FDG PET/CT scans with pre-marked lesions, a workflow to compute the final MTV was directly applied.

### (3) Tumor Burden Calculations

Baseline skull to mid-thigh+/−legs/whole body ¹⁸F-FDG PET/CT scans obtained prior to axi-cel were evaluated for MTV using custom tool implemented on MIM PACS version 6.8.4 (MIM Software Inc, Cleveland, Ohio). Briefly, lesions with PET SUV greater than the user selected liver reference, were automatically identified, such that voxels equal or greater than 41% of SUVmax of the lesion were selected to create a metabolically active region. Voxels over 41% SUVmax were included for calculation of lesion MTV (mL), as this was demonstrated most accurate for lymphoma. Additional details on the MTV process for automatic lesion selection are provided in Methods.

Following ¹⁸F-FDG PET/CT scan automatic marking, lesions were edited by a physician (EAD) and confirmed by a radiologist (HL or MSM). First, lesions due to physiologic FDG uptake (e.g. brain, bladder) and non-malignant lesions (e.g. degenerative disk disease) were removed (MTV-Semi-Automated). Then malignant lesions erroneously omitted were added and individual lesion contours were adjusted via the paintbrush tools to match tumor boundary precisely (MTV-Manual). Bone marrow tumor infiltration was included in the calculations. Reactive marrow due to chemical or physical stimulation was excluded. FIG. 12 shows an example of tumor lesion manipulation on an ¹⁸F-FDG PET/CT scan. 100. Available SPD (cm²) values and all SUVmax values were obtained from the clinical radiologist's report.

### (4) Statistical Analyses

MTV-Manual generated values were used for analysis. In cohort 1, Kendall's tau correlation coefficient was used to assess agreement in the ranked tumor burden values between tumor burden calculation techniques (MTV-Manual, MTV-Semi-Automated, SPD, and SUVmax).

Clinical outcomes were determined for subjects in each patient cohort (n=48) and compared between high versus low MTV group. The high and low tumor volume groups were selected based on the median MTV value in cohort 1. Once the MTV cutoff was validated on cohort 2 for OS and PFS, the two cohorts were combined for further comparison. Differences in patient attributes were investigated using chi square and Fischer's Exact test and non-parametric test of median.

OS and PFS were calculated from the time of axi-cel infusion until death, or progression, or the last date patient was known alive. Kaplan-Meier and log-rank test were used to assess differences in OS and PFS time estimates. Hazard ratios (HR) and 95% confidence intervals (CI) were reported. Univariate Cox regression analysis (UA) and multivariate Cox regression analysis (MVA), adjusting for variables that showed significant association with OS and PFS in UA and variables that were clinically relevant, were used to assess the differences in OS and PFS estimates between patients with high versus low volume tumors. These variables included bridging therapy (patients who received therapy versus those who did not) and LDH status before lymphodepleting chemotherapy (LDH>2×ULN versus <2×ULN). UA and MVA were also conducted for both OS and PFS for the entire patient population.

Overall response rate (ORR) included patients with a partial response and a complete response to therapy. Complete response rate (CR) was reported if achieved by last follow-up for each cohort. For the two patient cohorts combined, the association of ORR and CR with MTV group (low versus high) was evaluated and odds ratio (OR) and 95% CI were reported.

Maximum cytokine release syndrome (CRS) was graded by Lee criteria. Maximum neurotoxicity (NT) was graded by CTCAE v4.03. Any or G3-4 CRS and any or G3-4 NT were reported in each cohort. For the cohorts combined, the incidence of toxicities among patients across MTV groups was also evaluated and ORs with 95% CI were reported.

In all analyses, P-value <0.05 was defined as statistically significant. All statistical calculations were conducted using MedCalc Statistical Software version 19.1.5 (MedCalc Software by, Ostend, Belgium).

### b) Results

### (1) Patient Characteristics

For all 96 subjects in this study, patient and disease characteristics at the time of axi-cel treatment were obtained (Table 1). Bridging therapy was given to 31% of patients in cohort 1 and 65% of patients in cohort 2. Notably, only cohort 1 (46%) included patients enrolled on a prospective clinical trial, which prohibited the use of bridging therapy.

### (2) Tumor Burden Estimation Methods Comparison

On baseline ¹⁸F-FDG PET/CT scan, MTV was calculated for all 48 patients in cohort 1 and 48 patients in cohort 2 by two both MTV-Semi-Automated and MTV-Manual. Median time between baseline imaging and axi-cel infusion was 9 days (range, 6-46 days) for cohort 1 and 11 days (range, 0-91 days) for cohort 2. Tumor burden results are presented in Table 5.

TABLE 5
Tumor burden results by all calculation methods.

| | MTV-Manual, | MTV-Semi- | | |
|---|---|---|---|---|
| | mL | Automated, mL | SPD, cm² | SUVmax |
| | Median | Median | Median | Median |
| Cohort | (range) | (range) | (range) | (range) |
| Cohort 1 | 147.5 | 55.9 | 44.3 | 18.4 |
| | (4.2-1221.4) | (3.7-1104.6) | (4.3-336.7) | (7.4-42.1) |
| Cohort 2 | 72.8 | 35.62 | 43 | 19.5 |
| | (2.3-1275.3) | (0-652.6) | (23.2-240.2) | (1.8-53.2) |

MTV values and SUVmax were measured for all patients (n = 96). SPD was obtained for 35/48 patients in cohort 1 and 11/48 patients in cohort 2.

In cohort 1, comparison of tumor burden estimation methods showed that MTV-Manual had a positive correlation with MTV-Semi-Automated (correlation coefficient 0.61, 95% CI 0.39-0.74). However, there was no agreement between MTV-Manual or MTV-Semi-Automated with SPD (correlation coefficient 0.39, 95% CI 0.09-0.61; 0.32, 95% CI 0.06-0.53, respectively) or with SUVmax (correlation coefficient 0.14, 95% CI −0.06-0.33; 0.18, 95% CI −0.04-0.34, respectively).

### (3) High or Low Tumor Burden, Quantified by MTV, Associates with Progression-Free and Overall Survival

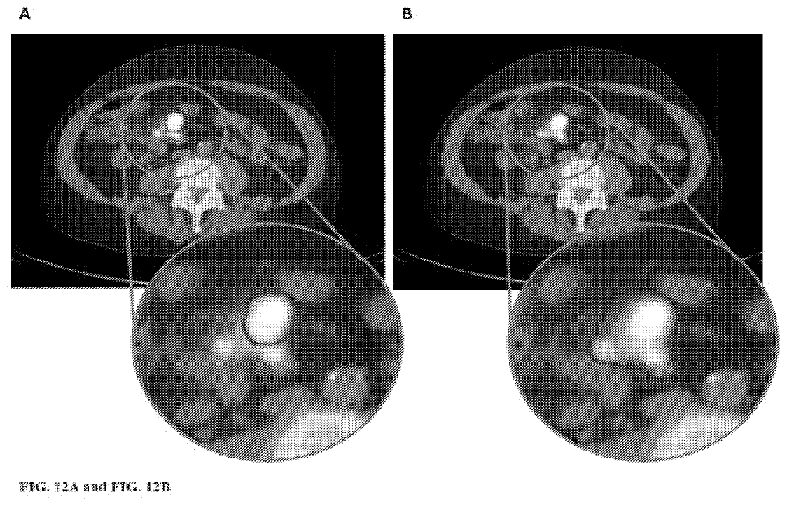

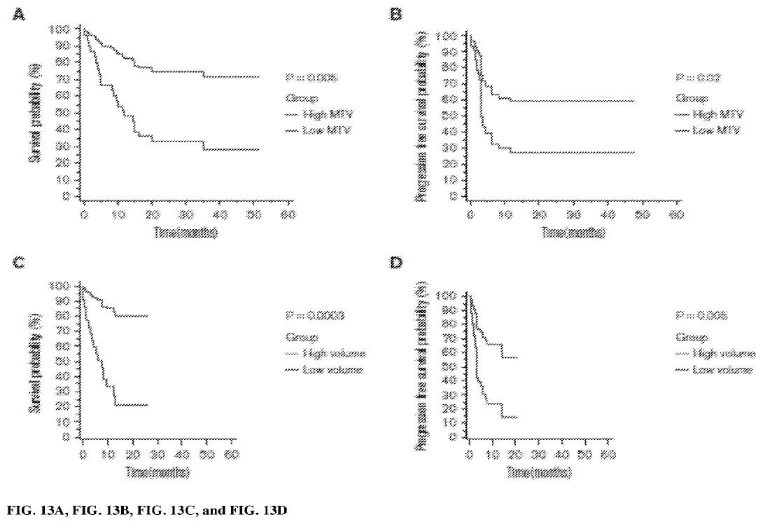

All endpoint analysis was performed using MTV-Manual values. In cohort 1, median OS was 34.98 months (95% CI 14.33-34.98) and median PFS was 5.85 months (95% CI 2.99-11.54). Median follow-up for survivors was 24.98 months (range, 10.59-51.02 months) (Table 1). Based on the median MTV value of 147.5 mL, patients were divided into a low (n=24) versus high (n=24) MTV group. Patients with low MTV had superior OS (HR=0.25, 95% CI 0.10-0.66) and PFS (HR=0.40, 95% CI 0.18-0.89) (FIGS. 13A and 13B). Bridging therapy did not associate with OS (HR=1.00, 95% CI 0.38-2.58) or PFS (HR=0.72, 95% CI 0.30-1.70). Similarly elevated LDH was not associated with OS (HR=1.45, 95% CI 0.56-3.73) or PFS (HR=1.04, 95% CI 0.42-2.60).

However, given the potential for bridging therapy impacting tumor burden and other potential confounding variables, we performed MVA considering receipt of bridging therapy, raised LDH>2×ULN before receiving conditioning chemotherapy, and MTV. On MVA, high MTV remained statistically significant for inferior OS (HR=0.20, 95% CI 0.07-0.57, P=0.002) and PFS (HR=0.30, 95% CI 0.12-0.72, P=0.007). Bridging therapy was not associated with OS (HR=1.02, 95% CI 0.39-2.69, P=0.95) or PFS (HR=0.78, 95% CI 0.32-1.87, P=0.58). Similarly, LDH status was not associated with OS (HR=0.60, 95% CI 0.21-1.68, P=0.33) or PFS (HR=0.51, 95% CI 0.18-1.43, P=0.20) (Table 6).

TABLE 6
Predicators of overall survival and progression free survival.

| | | | Univariate | Multivariate | Multivariate |
|---|---|---|---|---|---|
| | | | analysis | analysis | analysis |
| Cohort | Outcome | Variable | Odds ratio(95% CI) | Odds ratio(95% CI) | P value |
| Cohort 1 | Overall | MTV Manual | 0.25(0.10-0.66) | 0.20(0.07-0.57) | 0.002 |
| | Survival | High vs. low | | | |
| | | Bridging Therapy | 1.00(0.38-2.58) | 1.02(0.39-2.69) | 0.95 |
| | | Yes vs. No | | | |
| | | LDH before | 1.45(0.56-3.73) | 0.60(0.21-1.68) | 0.33 |
| | | conditioning | | | |
| | | >2xULN vs. | | | |
| | | <2xULN | | | |
| | Progression | MTV Manual | 0.40(0.18-0.89) | 0.30(0.12-0.72) | 0.007 |
| | free survival | High vs. low | | | |
| | | Bridging Therapy | 0.72(0.30-1.70) | 0.78(0.32-1.87) | 0.58 |
| | | Yes vs. No | | | |
| | | LDH before | 1.04(0.42-2.60) | 0.51(0.18-1.43) | 0.20 |
| | | conditioning | | | |
| | | >2xULN vs. | | | |
| | | <2xULN | | | |
| Cohort 2 | Overall | MTV Manual | 0.14(0.05-0.42) | 0.18(0.04-0.69) | 0.01 |
| | Survival | High vs. low | 2.99(0.85-10.43) | 0.77(0.15-3.84) | 0.75 |
| | | Bridging Therapy | | | |
| | | Yes vs. No | 5.86(2.18-15.71) | 3.17(1.07-9.40) | 0.03 |
| | | LDH before | | | |
| | | conditioning | | | |
| | | >2xULN vs. | | | |
| | | <2xULN | | | |
| | Progression | MTV Manual | 0.29(0.12-0.69) | 0.34(0.12-0.96) | 0.04 |
| | free survival | High vs. low | | | |
| | | Bridging Therapy | 2.00(0.78-5.14) | 0.93(0.29-2.93) | 0.93 |
| | | Yes vs. No | | | |
| | | LDH before | 4.22(1.59-11.22) | 3.18(1.09-9.31) | 0.03 |
| | | conditioning | | | |
| | | >2xULN vs. | | | |
| | | <2xULN | | | |

The model was then tested in cohort 2. In cohort 2, median OS was not reached and median PFS was 13.84 months (95% CI 3.94-13.84). Median follow-up for survivors was 12.03 months (range, 0.89-25.74 months). One patient was lost to follow up past 1 month (Table 1). The majority of the patients (n=30) were in the low MTV group, compared to high MTV (n=18), by the previously defined cutoff (147.5 mL). Patients with low MTV values were found to have superior OS (HR=0.14, 95% CI 0.05-0.42) and PFS (HR=0.29, 95% CI 0.12-0.69) (FIGS. 13C and 13D). Bridging therapy use was not statistically significant for inferior OS (HR=2.99, 95% CI 0.85-10.43) or PFS (HR=2.00, 95% CI 0.78-5.14). However, LDH status was associated with OS (HR=5.86, 95% CI 2.18-15.71) and PFS (HR=4.22, 95% CI 1.59-11.22).

On MVA after adjusting for bridging therapy use and LDH status, high MTV remained statistically significant for inferior OS (HR=0.18, 95% CI 0.04-0.69, P=0.01) and PFS (HR=0.34, 95% CI 0.12-0.96, P=0.04); bridging therapy use was not statistically significant for inferior OS (HR=0.77, 95% CI 0.15-3.84, P=0.75) or PFS (HR=0.93, 95% CI 0.29-2.93, P=0.93). LDH status was associated with OS (HR=3.17, 95% CI 1.07-9.40, P=0.03) and PFS (HR=3.18, 95% CI 1.09-9.31, P=0.03) (Table 6).

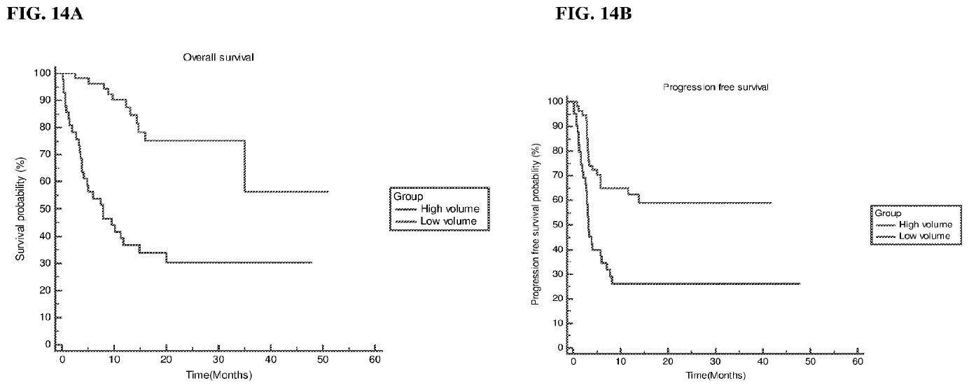

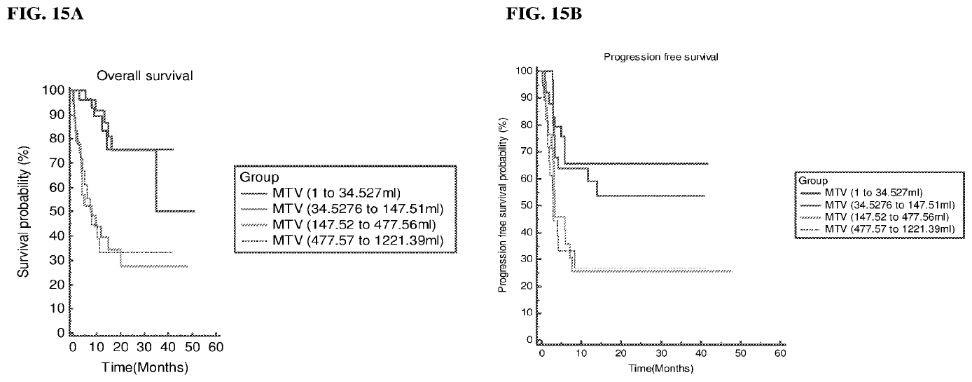

The model was applied to both cohorts (n=96) and confirmed the results (FIG. 14 and Table 7). To determine if there was a group with a low likelihood of benefitting from axi-cel, we evaluated MTV by quartiles. The two highest quartiles had similar OS and PFS (FIG. 15 and Table 8). Finally, the model was tested on a limited group of patients (n=72) that had a more stringently defined baseline PET, and results were similar to the main analysis (Table 9).

TABLE 7
Multivariate Model for cohort 1 and 2 combined (n = 96).

| | | | | Multivariate |
|---|---|---|---|---|
| | | Univariate analysis | Multivariate analysis | analysis P |
| | Variable | Odds ratio(95% CIs) | Odds ratio(95% CIs) | value |
| Overall Survival | MTV Manual (147.5 | 0.20(0.10 to 0.41) | 0.21(0.10 to 0.47) | 0.0001 |
| | cut off) | | | |
| | Bridging Therapy (yes | 1.45(0.77 to 2.76) | 1.17(0.61 to 2.24) | 0.61 |
| | vs. no) | | | |
| | Raised LDH before | 2.58(1.32 to 5.05) | 1.12(0.54 to 2.33) | 0.75 |
| | conditioning (greater | | | |
| | than 2x UNL vs. less | | | |
| | than 2X UNL) | | | |
| Progression Free | MTV Manual (147.5 | 0.36(0.20 to 0.63) | 0.35(0.18 to 0.67) | 0.001 |
| Survival | cut off) | | | |
| | Bridging Therapy (yes | 1.09(0.62 to 1.91) | 0.93(0.53 to 1.65) | 0.82 |
| | vs. no) | | | |
| | Raised LDH before | 1.79(0.93 to 3.45) | 0.98(0.47 to 2.04) | 0.97 |
| | conditioning | | | |
| | >2xULN vs. <2xULN | | | |

TABLE 8
Multivariate Model (with MTV quartiles) for cohort 1 and 2 combined (n = 96).

| | | Multivariate analysis | Multivariate |
|---|---|---|---|
| | Variable | Odds ratio(95% CIs) | analysis P value |
| Overall Survival | MTV Group (1 to 34.527 ml) | — | — |
| | MTV Group (34.5276 to 147.51 ml) | 1.20(0.36 to 3.96) | 0.76 |
| | MTV Group (147.52 to 477.56 ml) | 5.19(1.85 to 14.57) | 0.001 |
| | MTV Group (477.57 to 1221.39 ml) | 4.57(1.38 to 15.15) | 0.01 |
| | Bridging Therapy (yes vs. no) | 1.21(0.61 to 2.40) | 0.58 |
| | Raised LDH before conditioning | 1.16(0.53 to 2.55) | 0.69 |
| | >2xULN vs. <2xULN | | |
| Progression Free | MTV Group (1 to 34.527 ml) | — | — |
| Survival | MTV Group (34.5276 to 147.51 ml) | 1.39(0.58 to 3.29) | 0.45 |
| | MTV Group (147.52 to 477.56 ml) | 3.10(1.36 to 7.09) | 0.007 |
| | MTV Group (477.57 to 1221.39 ml) | 3.92(1.45 to 10.56) | 0.006 |
| | Bridging Therapy (yes vs. no) | 0.87(0.47 to 1.59) | 0.65 |
| | Raised LDH before conditioning | 0.91(0.41 to 1.97) | 0.81 |
| | >2xULN vs. <2xULN | | |

TABLE 9
Final Multivariate Model for patients with “true” baseline PET (n = 72).

| | | | Multivariate |
|---|---|---|---|
| | | Multivariate analysis | analysis |
| Outcome | Variable | Odds ratio (95% CIs) | P value |
| Overall | MTV Manual | 0.22(0.09 to 0.54) | 0.001 |
| survival | High vs. low | | |
| | Bridging Therapy | 1.39(0.65 to 2.96) | 0.39 |
| | Yes vs. No | | |
| | LDH before conditioning | 0.80(0.32 to 2.01) | 0.64 |
| | >2xULN vs. <2xULN | | |
| Progression | MTV Manual | 0.37(0.17 to 0.79) | 0.001 |
| free | High vs. low | | |
| survival | Bridging Therapy | 1.03(0.53 to 2.01) | 0.92 |
| | Yes vs. No | | |
| | LDH before conditioning | 0.86(0.34 to 2.14) | 0.74 |
| | >2xULN vs. <2xULN | | |

72 out of 96 patients fit the following criteria: no bridging therapy, or only steroids as bridging, or bridging and baseline PET done afterwards. In addition, patients must have had baseline PET within 28 days from start of conditioning chemotherapy (one patient had a baseline scan the day of CAR T-cell infusion).

### (4) Response Rates in Low Versus High Tumor Burden Patients

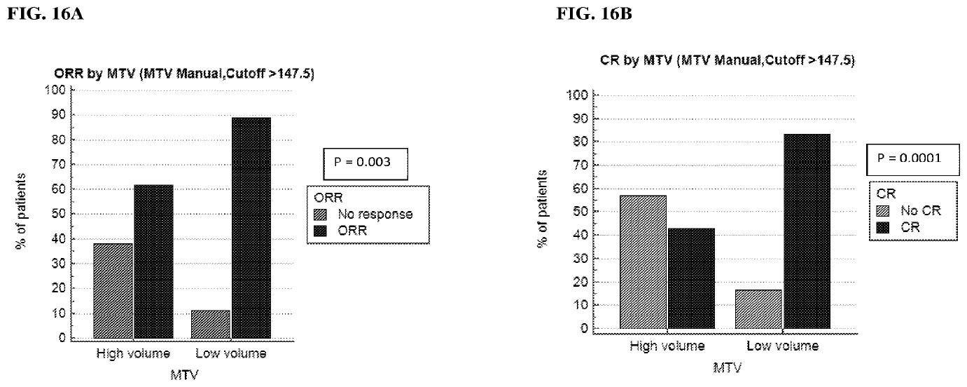

Response rates per cohort are summarized in Table 1. In the two cohorts combined 74 out of 96 patients had a response (77% ORR) and 63 out of 96 patients had CR (65.6%) to axi-cel. Utilizing the model in the combined cohorts, low MTV was shown to be associated with superior ORR (OR=4.92, 95% CI 1.71-14.10, P=0.003) and CR (OR=6.66, 95% CI 2.60-17.08, P <0.0001) (FIGS. 16A and 16B).

### (5) Toxicity in Low Versus High Tumor Burden Patients

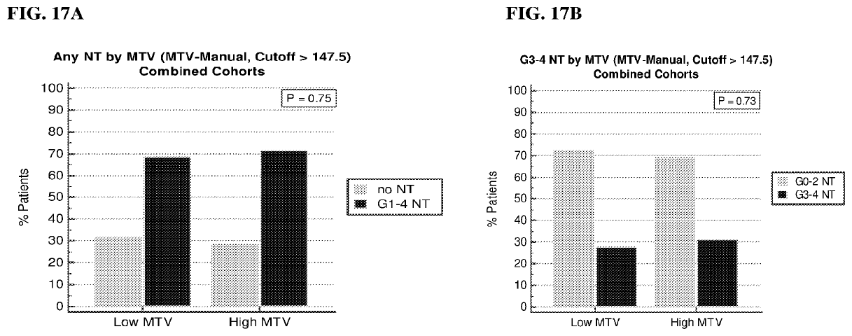

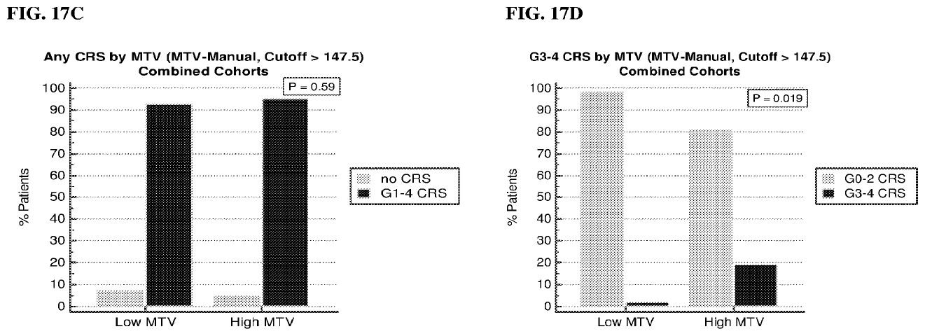

Toxicity rates per cohort are summarized in Table 1. In the two cohorts combined, 67 out of 96 patients (69.7%) had any grade (G) NT and 28 out of 96 patients (29.1%) had G 3-4 NT; and 90 out of 96 patients (93.7%) had any grade CRS and 9 out of 96 patients (9.3%) had G3-4 CRS. No G5 NT or CRS were observed in any patients. Applying the model to the combined cohorts, high MTV was not associated with any grade NT or G3-4 NT (OR=1.14, 95% CI 0.47-2.77, P=0.75; OR=1.16, 95% CI 0.48-2.82, P=0.73, respectively) (FIGS. 17A and 17B) and it was not associated with any grade CRS (OR=1.6, 95% CI 0.27-9.18, P=0.59). However, high grade tumor burden by MTV was associated with G3-4 CRS (OR=12.4, 95% CI 1.49-104.2, P=0.019) (FIGS. 17C and 17D).

### c) Discussion

This study establishes the association of MTV on baseline ¹⁸F-FDG PET/CT with OS and PFS in patients with refractory/relapsed LBCL treated with axi-cel using two separate cohorts of patients. There was lack of agreement between MTV derived by either of two different MTV calculation methods, MTV-Semi-Automated and MTV-Manual, and SPD or SUVmax, which was expected considering the difference in extent and type of measurement. The manually and semi-automated MTV values correlated, however there was considerable variation in the estimated MTV depending on method. We, a priori, considered the MTV-Manual method to be superior as it clearly abrogates the exclusion of some tumor areas using the MTV-Semi-Automated method (FIG. 12). Therefore, the model was constructed using MTV-Manual values.

Due to the relatively limited number of patients in cohort 1, median MTV was chosen as the cutoff. Although this value was lower than the reported general range for DLBCL, and close to those for LBCL patients receiving CAR T-Cell therapy, it is difficult to compare MTV values calculated using different methods (Table 5). For example, pre-tisagenlecleucel MTV in JULIET, the cutoff was 100 mL, determined by median MTV via MIM automatic methodology. Alternatively, Wang et al. selected a cutoff of 72 cm³, as determined by median MTV, in their CAR T-cell study.

Given the more frequent use of bridging therapy in the second cohort, we performed MVA and included receipt of bridging. Importantly, the relationship between MTV and OS and PFS did not change based on bridging therapy use and status of LDH before conditioning. Combining both cohorts, the low MTV group had significantly higher ORR and CR. Additionally, high baseline MTV was prognostic of G3-4 CRS by Lee criteria, but not of any CRS, or any NT or G3-4 NT by CTCAE criteria. The efficacy findings was validated in a subgroup of the patients that had scans within 28 days of starting conditioning chemotherapy (one patient had a baseline scan the day of CAR T-cell infusion) and excluding those without post bridging scans.

It was found upon analysis of PFS and OS by MTV quartile, that the two highest MTV quartiles had similarly worse outcomes. It is unclear why tumor burden appears to impact efficacy when it reaches a certain threshold, rather than in a continuous manner. Despite the worse outcomes, a subset of patients with high tumor burden have ongoing remissions indicating that high tumor burden alone does not preclude a patient from receiving axi-cel.

Since CAR T-cell therapy is relatively new, a very limited number of investigations exist on the subject of tumor burden, especially calculated by MTV. In the JULIET and TRANSCEND studies regarding two different types of CAR T-cell therapy (CTL019 and JCAR017, respectively), higher tumor burden was associated with greater toxicity. In the JULIET post hoc analysis including 95 treated relapsed/refractory DLBCL patients, high MTV was associated with G3-4 CRS by Lee criteria but not any NT or G3-4 NT by CTCAE. In a smaller study of 19 patients with refractory/relapsed non-Hogkin lymphoma treated with CAR T-cell therapy, after a median follow up of 5 months, calculated baseline MTV did not differ significantly between patients who had responded and those who had not (P=0.62) and MTV was not significantly associated with OS (P=0.67). Lower MTV was significantly associated with mild and moderate G0-2 CRS compared to G3-4 CRS (P=0.008). In agreement with these studies, the results showed an association of MTV with G3-4 CRS, but not NT by CTCAE. However, MTV was found to be associated with survival and response to therapy.

The main strengths of this study are the methodically derived manual baseline MTV values and the successful validation in a separate cohort, both of which increase validity of the results demonstrating that tumor burden impacts efficacy outcomes. The reproducibility of MTV-Manual values can be achieved using metabolic tumor volume calculating software by both non-radiology and radiology physicians, however we encourage manual resizing of lesions. Processing time per scan was not captured, however it is believed that MTV-Manual takes longer than MTV-Semi-Automated due to the additional processing steps. Anecdotally, we found the time per Manual scan was highly variable depending on the number, size, and complexity of lesions. Further optimization to improve the efficiency and accuracy of automated tumor contouring can reduce the time for, or entirely eliminate the need for, manual processing.

This study is limited by its retrospective nature and the low number of subjects available due to the novelty of the therapy. Notably, there were differences in the two patient cohorts: they varied in terms of follow-up time, secondary R-IPI scores, and use of bridging therapy, which itself can have had a confounding effect on both outcomes and on tumor burden in the case where PET scans were done before bridging. Additionally, American Society for Transplantation and Cellular Therapy (ASTCT) grading was not captured in a majority of the patients, although the high concordance of neurotoxicity grade 3-4 by CTCAE and ASTCT was reported.

In conclusion, this study showed that tumor burden, measured by MTV on baseline ¹⁸F-FDG PET/CT scan, is associated with PFS and OS in axi-cel treated LBCL patients. Since patients with lower tumor burden had superior survival and response rates, immunotherapeutic regimens should employ tumor debulking prior to CAR T-cell therapy or earlier referral for therapy in order to improve clinical outcomes in this group of patients.

### D. REFERENCES

Aerts H J, Velazquez E R, Leijenaar R T, et al: Decoding tumour phenotype by noninvasive imaging using a quantitative radiomics approach. Nat Commun 5:4006, 2014
Ahmadzadehfar H, Rodrigues M, Zakavi R, Knoll P, Mirzaei S. Prognostic significance of the standardized uptake value of pre-therapeutic (18)F-FDG PET in patients with malignant lymphoma. Med Oncol. 2011; 28(4):1570-6.
Ahmadzadehfar H, Rodrigues M, Zakavi R, Knoll P, Mirzaei S. Prognostic significance of the standardized uptake value of pre-therapeutic (18)F-FDG PET in patients with malignant lymphoma. Med Oncol. 2011; 28(4):1570-6.
Balagurunathan Y, Gu Y, Wang H, et al: Reproducibility and Prognosis of Quantitative Features Extracted from CT Images. Transl Oncol 7:72-87, 2014
Balagurunathan Y, Kumar V, Gu Y, et al: Test-retest reproducibility analysis of lung CT image features. J Digit Imaging 27:805-23, 2014
Borchmann P, Tam C, Jager U, et al. An updated analysis of JULIET, a global pivotal Phase 2 trial of tisagenlecleucel in adult patients with relapsed or refractory (r/r) diffuse large b-cell lymphoma (DLBCL) [abstract]. HemaSphere. 2018; 2(S1). Abstract S799.
Cheson B D, Fisher R I, Barrington S F, et al. Recommendations for initial evaluation, staging, and response assessment of Hodgkin and non-Hodgkin lymphoma: the Lugano classification. J Clin Oncol. 2014; 32(27):3059-3068.
Cheson B D. The International Harmonization Project for response criteria in lymphoma clinical trials. Hematol Oncol Clin North Am. 2007; 21(5):841-854.
Fischer A. FDA approves CAR-T cell therapy to treat adults with certain types of large B-cell lymphoma. Oct. 18, 2017.
Gatenby R A, Grove O, Gillies R J: Quantitative imaging in cancer evolution and ecology. Radiology 269:8-15, 2013
Gillies R J, Kinahan P E, Hricak H: Radiomics: Images Are More than Pictures, They Are Data. Radiology 278:563-77, 2016
Gu Y, Kumar V, Hall L O, et al: Automated Delineation of Lung Tumors from CT Images Using a Single Click Ensemble Segmentation Approach. Pattern Recognit 46:692-702, 2013
Guo B, Tan X, Ke Q, Cen H. Prognostic value of baseline metabolic tumor volume and total lesion glycolysis in patients with lymphoma: A meta-analysis. PLoS One. 2019; 14(1):e0210224.
Ilyas H, Mikhaeel N G, Dunn J T, et al. Defining the optimal method for measuring baseline metabolic tumour volume in diffuse large B cell lymphoma. Eur J Nucl Med Mol Imaging. 2018; 45(7):1142-1154.
Jain M D, Bachmeier C A, Phuoc V H, Chavez J C. Axicabtagene ciloleucel (KTE-C19), an anti-CD19 CAR T therapy for the treatment of relapsed/refractory aggressive B-cell non-Hodgkin's lymphoma. Ther Clin Risk Manag. 2018; 14:1007-1017.
Kalpathy-Cramer J, Mamomov A, Zhao B, et al: Radiomics of Lung Nodules: A Multi-Institutional Study of Robustness and Agreement of Quantitative Imaging Features. Tomography: a journal for imaging research 2:430-437, 2016
Kalpathy-Cramer J, Zhao B, Goldgof D, et al: A Comparison of Lung Nodule Segmentation Algorithms: Methods and Results from a Multi-institutional Study. Journal of digital imaging: 1-12, 2016
Kumar V, Gu Y, Basu S, et al: Radiomics: the process and the challenges. Magn Reson Imaging 30:1234-48, 2012
Lee D W, Gardner R, Porter D L, et al. Current concepts in the diagnosis and management of cytokine release syndrome. Blood. 2014; 124(2):188-95.
Lee D W, Santomasso B D, Locke F L, et al. ASTCT Consensus Grading for Cytokine Release Syndrome and Neurologic Toxicity Associated with Immune Effector Cells. Biol Blood Marrow Transplant. 2019 April; 25(4):625-638.
Leijenaar R T, Nalbantov G, Carvalho S, et al: The effect of SUV discretization in quantitative FDG-PET Radiomics: the need for standardized methodology in tumor texture analysis. Sci Rep 5:11075, 2015
Liu Y, Wang H, Li Q, et al: Radiologic Features of Small Pulmonary Nodules and Lung Cancer Risk in the National Lung Screening Trial: A Nested Case-Control Study. Radiology 286:298-306, 2018
Locke F L, Ghobadi A, Lekakis L J, et al. Outcomes by prior lines of therapy in ZUMA-1, the pivotal phase 2 study of axicabtagene ciloleucel (axi-cel) in patients with refractory large B-cell lymphoma [abstract]. J Clin Oncol. 2018; 36(15 suppl). Abstract 3039.
Locke F L, Neelapu S S, Bartlett N L, et al. Phase 1 results of ZUMA-1: a Multicenter Study of KTE-C19 Anti-CD19 CAR T cell therapy in refractory aggressive lymphoma. Mol Ther. 2017; 25(1):285-295.
Malek E, Sendilnathan A, Yellu M, Petersen A, Fernandez-Ulloa M, Driscoll J J. Metabolic tumor volume on interim PET is a better predictor of outcome in diffuse large B-cell lymphoma than semiquantitative methods. Blood Cancer Journal. 2015; 5:e326.
Maziarz R T, Schuster S, Ericson S, et al. Cytokine release syndrome and neurotoxocity by baseline tumor burden in adults with relapsed or refractory diffuse large B-cell lymphoma treated with tisagenleucleucel [abstract]. Hematological Oncology. 2019; 37(S2): 307. Abstract 253.
Maziarz R T, Schuster S J, Romanov V V, et al. Grading of neurological toxicity in patients treated with tisagenlecleucel in the JULIET trial. Blood Adv. 2020; 4(7):1440-1447.
Meignan M, Sasanelli M, Casasnovas R O, et al. Metabolic tumour volumes measured at staging in lymphoma: methodological evaluation on phantom experiments and patients. Eur. J. Nucl. Med. Mol. Imaging. 2014; 41:1113-1122.
National Cancer Institute, National Institutes of Health, U.S. Department of Health and Human Services. Common Terminology Criteria for Adverse Events (CTCAE) Version 4.0. Published May 28, 2009; Revised Version 4.03 Jun. 14, 2010.
O J H, Lodge M A, Wahl R L. Practical PERCIST: A Simplified Guide to PET Response Criteria in Solid Tumors 1.0. Radiology. 2016; 280(2):576-84.
Pennisi M, Jain T, Santomasso B D, et al. Comparing CAR T-cell toxicity grading systems: application of the ASTCT grading system and implications for management. Blood Adv. 2020; 4(4):676-686.
Schabath M B, Massion P P, Thompson Z J, et al: Differences in Patient Outcomes of Prevalence, Interval, and Screen-Detected Lung Cancers in the CT Arm of the National Lung Screening Trial. PLoS One 11:e0159880, 2016
Shafiq-Ul-Hassan M, Zhang G G, Latifi K, et al: Intrinsic dependencies of CT radiomic features on voxel size and number of gray levels. Med Phys 44:1050-1062, 2017
Siddiqi T, Abramson J S, Li D, et al. Patient characteristics and pre-infusion biomarkers of inflammation correlate with clinical outcomes after treatment with the defined composition, CD19-targeted CAR T cell product, JCAR017. Blood. 2017; 130: 193.
Song M K, Chung J S, Shin H J, et al. Clinical value of metabolic tumor volume by PET/CT in extranodal natural killer/T cell lymphoma. Leukemia Res. 2013; 37:58-63.
Song M K, Yang D H, Lee G W, et al. High total metabolic tumor volume in PET/CT predicts worse prognosis in diffuse large B cell lymphoma patients with bone marrow involvement in rituximab era. Leuk Res. 2016; 42:1-6.
Wahl R L, Jacene H, Kasamon Y, Lodge M A. From RECIST to PERCIST: Evolving Considerations for PET response criteria in solid tumors. J Nucl Med. 2009; 50:122S-50S.
Wang J, Hu Y, Yang S, et al. Role of Fluorodeoxyglucose Positron Emission Tomography/Computed Tomography in Predicting the Adverse Effects of Chimeric Antigen Receptor T Cell Therapy in Patients with Non-Hodgkin Lymphoma. Biol Blood Marrow Transplant. 2019; 25(6):1092-1098.
Xie M, Wu K, Liu Y, Jiang Q, Xie Y. Predictive value of F-18 FDG PET/CT quantization parameters in diffuse large B cell lymphoma: a meta-analysis with 702 participants. Medical Oncology (Northwood, London, England). 2014; 32(1):446.
Zhao B, Tan Y, Tsai W-Y, et al: Reproducibility of radiomics for deciphering tumor phenotype with imaging. Scientific Reports 6:23428, 2016.

1. A method of assaying the response of a cancer in a patient to immune-based or targeted therapy, comprising: measuring tumor burden prior to administration of the immune-based or targeted therapy to create a baseline tumor burden; wherein a high baseline tumor burden indicates that the patient will have a decreased, less efficacious, and/or less durable response. response to immune-based or targeted therapy.

2. The method of assaying the response of a cancer in a patient to immune-based or targeted therapy of claim 1, wherein the tumor burden is measured by measuring total metabolic tumor volume.

3. The method of assaying the response of a cancer in a patient to immune-based or targeted therapy of claim 1, wherein tumor burden is measured 1, 2, 3, 4, 5, 6, 7, 8, 9, 10, 11, 12, 13, 14, 15, 16, 17, 19, 20, 21, 22, 23, 24, 25 or more additional times after administration of the immune-based or targeted therapy.

4. The method of assaying the response of a cancer in a patient to immune-based or targeted therapy of claim 1, wherein total metabolic tumor volume is measured manually.

5. The method of assaying the response of a cancer in a patient to immune-based or targeted therapy of claim 1, wherein total metabolic tumor volume is measured by functional imaging that measures glucose uptake.

6. The method of assaying the response of a cancer in a patient to immune-based or targeted therapy of claim 5, wherein total metabolic tumor volume is measured by positron emission tomography (PET).

7. The method of assaying the response of a cancer in a patient to immune-based or targeted therapy of claim 1, wherein a metabolic tumor volume greater than 130 mL is a high baseline tumor burden.

8. The method of assaying the response of a cancer in a patient to immune-based or targeted therapy of claim 1, wherein the immune based or targeted therapy comprises an immunodepleting therapy and a CAR T cell infusion.

9. The method of assaying the response of a cancer in a patient to immune-based or targeted therapy of claim 8, wherein the CAR T cell infusion comprises an anti-CD19 CAR T cell infusion.

10. A method of treating a cancer in a subject comprising measuring tumor burden prior to administration of the immune-based or targeted therapy to create a baseline tumor burden; wherein a high baseline tumor burden indicates that the patient will have a decreased, less efficacious, and/or less durable response to immune-based or targeted therapy relative to a control; wherein a low baseline tumor burden a patient will have an efficacious response to immune-based or targeted therapy relative to a control; and administering to a patient with a low baseline tumor burden an immune-based or targeted therapy or administering to a patient with a high baseline tumor burden a chemotherapeutic agent, a higher dose of immune-based or targeted therapy, or multiple rounds of immune-based or targeted therapy.

11. The method of treating a cancer in a subject of claim 10, wherein the tumor burden is measured by measuring total metabolic tumor volume.

12. The method of treating a cancer in a subject of claim 10, wherein a metabolic tumor volume greater than 130 mL is a high baseline tumor burden.

13. The method of treating a cancer in a subject of claim 10, further comprising measuring tumor burden 1, 2, 3, 4, 5, 6, 7, 8, 9, 10, 11, 12, 13, 14, 15, 16, 17, 19, 20, 21, 22, 23, 24, 25 or more additional times following administration of the immune-based or targeted therapy.

14. The method of treating a cancer in a subject of claim 10, wherein total metabolic tumor volume is measured manually.

15. The method of treating a cancer in a subject of claim 10, wherein total metabolic tumor volume is measured by functional imaging that measures glucose uptake.

16. The method of treating a cancer in a subject of claim 15, wherein total metabolic tumor volume is measured by positron emission tomography (PET).

17. The method of treating a cancer in a subject of claim 10, wherein the immune based or targeted therapy comprises an immunodepleting therapy and an autologous CAR T cell infusion.

18. The method of treating a cancer in a subject of claim 17, wherein the CAR T cell infusion comprises an anti-CD19 CAR T cell infusion.
